(19) United States
(12) Patent Application Publication
(10) Pub. No.: US 2003/0190320 A1
(43) Pub. Date: Oct. 9, 2003

(54) Monoclonal antibodies that are cross-reactive against bacterial collagen binding proteins

(76) Inventors: Pietro Speziale, Pavia (IT); Livia Visai, Rosate (IT); Giampiero Pietrocola, Pavia (IT)

Correspondence Address: LARSON & TAYLOR, PLC, 1199 NORTH FAIRFAX STREET, SUITE 900, ALEXANDRIA, VA, 22314, US

(21) Appl. No.: 10/370,100

(22) Filed: Feb. 21, 2003

Related U.S. Application Data
(60) Provisional application No. 60/361,347, filed on Mar. 5, 2002.
(60) Provisional application No. 60/357,832, filed on Feb. 21, 2002.

(51) Int. Cl. A61K 39/40; G01N 33/554; G01N 33/569; C07K 16/12

(52) U.S. Cl. 424/165.1; 435/7.32; 530/388.4

(57) ABSTRACT

Cross-reactive monoclonal antibodies are provided which are generated from peptides from Enterococcus faecalis, including the ACE40 and the ACE19 protein, and the CNA19 peptide from Staphylococcus aureus, and which can bind to the collagen-binding proteins from bacteria from a variety of species including enterococcal bacteria, staphylococcal bacteria and streptococcal bacteria. These monoclonal antibodies may then be formed into suitable pharmaceutical compositions, and they are thus particularly effective in providing methods of treating or preventing bacterial infections from a wide range of bacterial species.

### CROSS REFERENCE TO RELATED APPLICATIONS

The present application claims the benefit of U.S. provisional applications Ser. No. 60/361,347, filed Mar. 5, 2002, and Ser. No. 60/357,832, filed Feb. 21, 2002.

### FIELD OF THE INVENTION

The present invention relates in general to monoclonal antibodies that have been generated against collagen binding proteins and peptides from bacteria such as Staphylococcus aureus, Enterococcus faecalis, and Enterococcus faecium, as well as streptococcal bacteria, and in particular to monoclonal antibodies against certain peptide fragments from the collagen binding domains from these proteins such as ACE19 and ACE40 which evidence cross-reactivity across species, as well as their use in treating or preventing bacterial infections.

### BACKGROUND OF THE INVENTION

The magnitude of gram-positive nosocomial infections has been documented extensively in both the scientific literature as well as in the lay press over the past two decades. Since staphylococci account for the single largest cause of nosocomial infections they have been the focus of most reports. Traditionally, the generic antibiotic vancomycin has been the drug of choice to treat gram-positive infections. However, the continued rise in the prevalence of methicillin resistant S. aureus (MRSA) and the emergence of vancomycin resistant isolates of S. aureus from intensive care units from around the world has served as a rallying point for the clinical community, biopharmaceutical companies, and governmental agencies to develop novel therapeutics. The continued overuse of vancomycin has not only led to the development of resistant S. aureus strains, but it has also resulted in the emergence of resistant strains of enterococci. In 1986, the first clinical isolates of Enterococcus faecium were reported in France. A decade later, vancomycin-resistant enterococci (VRE) have reported in 18 countries and 6 continents. The problem in the United States is extremely troublesome, were >20% of enterococcal isolates reported in the 1998 report of the National Nosocomial Infectious Surveillance System (NNIS) hospitals were vancomycin resistant. The resistant rate was >50% higher than that reported for the same hospitals from the period 1993-1997. Enterococci now account for 10% of all nosocomial bloodstream and 20% of cardiovascular infections in the U.S. Moreover, VRE tend to be concomitantly resistant to moderate to high levels of penicillins and aminoglycosides and therefore must be treated with unproven combinations of antibiotics. Even with the recent introductions of linezolid and quinupristin/dalfopristin for the treatment of certain types of VRE infections, a significant gap in the therapeutic armamentarium of the clinician exists. These data indicate that the development of novel therapies that can prevent infection in a prophylactic manner or enhance current treatment modalities or are warranted.

In the U.S., VRE infections are typically seen in moderately to severely ill patients. Therefore, it makes sense that most data detailing the VRE infections come from acute-care hospitals, specifically ICUs, oncology wards, and transplantation units. Host factors attributed with VRE infections include advanced age, APACHE score, neutronpenia, hematological malignancy, and prior nosocomial infection. Prolonged antibiotic exposure to vancomycin has also been associated as a risk factor for VRE infection. The most significant risk factors are length of hospital stay, proximity to a patient colonized or infected with VRE, and severe underlying illness.

While a considerable amount of data is available on host or environmental conditions that influence the acquisition of a VRE infection, little is know about the virulence mechanisms of the pathogen. The sophisticated interplay between host and bacterium is still not understood, however, successful colonization is presumed to be the defining event leading to initiation of an infection. MSCRAMM® (Microbial Surface Components Recognizing Adhesive Matrix Molecules) proteins are a family of cell-surface adhesins that recognize and specifically bind to distinct extracellular components of host tissues or to serum-conditioned implanted biomaterials such as catheters, artificial joints, and vascular grafts. Once an organism has successfully adhered and colonized host tissues, expression of specific genes may be altered contributing to a phenotype that is more resistant to antimicrobials. Therefore intervention that impacts early events in the infectious process may lead to a beneficial clinical outcome. MSCRAMM® proteins provide an excellent target for immunological attack by antibodies. Antibodies against MSCRAMM® proteins exhibit at least two biological properties. Initially, the highly specific antibodies prevent microbial adherence as well as recolonization of host tissues or biomaterials. Secondly, the increased level of MSCRAMM® protein antibodies bound to the bacterial cell wall facilitate a rapid clearance of the organism through opsonophagocytosis.

Earlier work has showed that the presence of a collagen-binding MSCRAMM® protein, CNA, is necessary and sufficient to allow S. aureus to adhere to collagenous tissues such cartilage (7). Furthermore, a S. aureus strain that normally lacks the cna gene becomes more virulent in experimental septic arthritis model, when the cna gene is introduced (8). Immunization with collagen adhesin from S. aureus and passive transfer of collagen-adhesin specific antibodies protected mice against S. aureus mediated septic death (9). In a recent study a panel of 22 monoclonal antibodies were raised against CNA19 and characterized. All mAbs appeared to recognize conformational-dependent epitopes and inhibited ¹²⁵I-collagen binding to CNA19 as well as to cells of S. aureus. Furthermore, some of the mAbs could effectively displace 125I-collagen bound to bacteria or detach bacteria that had adhered to a collagen substrate. This finding raises the possibility that these mAbs may be used as therapeutic agents (10).

Recent studies identified a gene from E. faecalis that codes for a MSCRAMM® protein designated ACE (adhesin of collagen from enterococci) that has the property to bind collagen (11). ACE has a structural organization similar to that of CNA and contains an N-terminal signal peptide, a collagen-binding region A followed by the B regions composed of repeated units and in the C-terminus an element required for cell wall anchoring, a transmembrane domain and a short cytoplasmic tail (12). A central region (aa 174-319), ACE19, in the A domain (ACE40, aa 32-367) of E. faecalis ACE has a high degree of sequence similarity to residues 151-318 of S. aureus CNA protein. Within this span of amino acids 27% of the residues are identical to residues in CNA19 and an additional 29% are similar. Significant similarity (46%) continues throughout the A domain of ACE and the corresponding region of the CNA domain; beyond the A domain, there is no sequence homology between ACE and CNA.

It thus remains a challenge to identify and utilize the information concerning MSCRAMM® proteins to develop monoclonal antibodies which cross-react between species of pathogenic organisms. Such cross-reactive antibodies are highly desirable because they can be used in treating or preventing a much wider range of bacterial infections and thus be effective in a greater range of therapeutic applications.

### SUMMARY OF THE INVENTION

Accordingly, it is an object of the present invention to provide cross-reactive monoclonal antibodies to collagen binding proteins and peptides from Enterococcal and Staphylococcal surface proteins that can be used to treat or prevent a wide variety of bacterial infections such as those caused by staphylococcal and streptococcal bacteria in addition to enterococcal bacteria.

It is also an object of the present invention to provide cross-reactive monoclonal antibodies which are generated from enterococcal peptides from the ACE protein such as ACE19 and ACE40 from E. faecalis.

It is also an object of the present invention to provide cross-reactive monoclonal antibodies which are generated from peptides from staphylococcal collagen binding proteins such as the CNA19 and CNA55 peptides from the CNA protein from S. aureus.

It is a further object of the present invention to provide cross-reactive antibodies and antisera which can recognize the collagen-binding proteins from a variety of bacteria, including the staphylococcal CNA protein in addition to the enterococcal ACE protein, and which can thus be useful in methods of treating, preventing, identifying or diagnosing a variety of bacterial infections.

These and other objects are provided by virtue of the present invention which comprises the isolation, purification and/or use of cross-reactive monoclonal antibodies which are generated against and which can recognize the effective regions of the ACE protein and/or its binding subdomains, including the peptide regions identified as ACE19 and ACE40, or which are generated from the CNA19 or CNA55 peptides, for the prevention and treatment of infections caused by bacteria such as staphylococcal and streptococcal bacteria in addition to enterococcal bacteria. As set forth herein, the cross-reactive antibodies of the present invention have been shown to recognize epitopes from more than one species of bacteria and thus can be utilized to develop compositions and vaccines to treat or protect against a wider variety of bacterial infections.

These embodiments and other alternatives and modifications within the spirit and scope of the disclosed invention will become readily apparent to those skilled in the art from reading the present specification and/or the references cited herein, all of which are incorporated by reference.

### BRIEF DESCRIPTION OF THE DRAWING FIGURES

FIG. 1 is a graphic representation of the binding properties of an anti-ACE40 monoclonal antibody to E. faecium.

FIG. 2 is a graphic representation of the binding properties of an anti-ACE19 monoclonal antibody to E. faecium.

FIG. 3 is a graphic representation of the binding properties of an anti-ACE40 monoclonal antibody to E. faecalis.

FIG. 4 is a graphic representation of the binding properties of an anti-ACE monoclonal antibody to cells of Streptococcus pyogenes.

FIG. 5 is a graphic representation of the binding properties of an anti-CNA19 monoclonal antibody to cells of E. faecium.

FIG. 6 is a graphic representation of the binding properties of an anti-CNA55 monoclonal antibody to cells of E. faecium.

FIG. 7 is a graphic representation of the binding properties of an anti-CNA19 and CNA55 monoclonal antibodies to cells of Streptococcus pyogenes.

FIG. 8 is a graphic representation of cross-reactivity of mAbs generated against ACE with a recombinant collagen-binding protein from E. faecium.

FIG. 9 is a graphic representation of cross reactivity of mAbs generated against CNA19 with a recombinant collagen-binding protein from E. faecium.

### DETAILED DESCRIPTION OF THE PREFERRED EMBODIMENTS

In accordance with the present invention, there are provided cross-reactive monoclonal antibodies that have been generated from regions of the ACE protein, which is a 74 kDa protein which has a structural similarity to that of MSCRAMM® proteins from other Gram-positive bacteria. The ACE protein of the invention is an extracellular matrix-binding protein of enterococcal bacteria such as Enterococcus faecalis, which can bind with collagens such as collagen type I and type IV and with laminin, and has the sequence as disclosed in PCT publication, WO00/68242, incorporated herein by reference. The collagen-binding Ace protein from Enterococcus faecalis has the amino acid sequence set forth herein as SEQ ID No. 1, and the nucleic acid sequence encoding the Ace protein is set forth herein as SEQ ID No. 2.

As discussed further herein, certain regions of the ACE protein have been identified, and these include regions known as the A domain (or ACE40) at amino acids 32-367 of the E. faecalis ACE protein, and fragment ACE19 located at amino acids 174-319 of the ACE protein. In accordance with the present invention, there are provided monoclonal antibodies which are generated from and which can recognize can bind to ACE A domain (ACE40) and ACE19 fragment, and these generated monoclonal antibodies have been isolated and purified by the present inventors and shown to have cross-reactive properties as set forth below. The monoclonal antibodies in accordance with the invention can be used to treat or protect against infections by enterococcal and staphylococcal bacteria as discussed further below. In addition, there are provided cross-reactive monoclonal antibodies that have been generated from regions of the CNA protein, another MSCRAMM® protein from the Gram-positive bacteria, Staphylococcus aureus. The collagen binding protein identified as CNA has been disclosed, e.g., in WO97/43314, incorporated herein by reference, and monoclonal cross-reactive antibodies of the invention are generated from fragments such as CNA19, amino acids 151-318 of the CNA protein. As set forth below, these cross-reactive monoclonal antibodies can be used to treat or prevent a wide variety of bacterial infections.

In accordance with the invention, the cross-reactive monoclonal antibodies of the invention may be prepared in a number of suitable ways that would be well known in the art, such as the well-established Kohler and Milstein method described above which can be utilized to generate monoclonal antibodies. In one such suitable method, mice are injected intraperitoneally once a week for a prolonged period with a purified recombinant protein such as ACE40, ACE19 or CNA19 as described above, followed by a test of blood obtained from the immunized mice to determine reactivity to the purified protein or fragment. Following identification of mice reactive to the tested protein, lymphocytes isolated from mouse spleens are fused to mouse myeloma cells to produce hybridomas positive for the antibodies against these proteins which are then isolated and cultured, following by purification and isotyping.

As described, for example, in J. Biol. Chem. 1999, 274, 26939-26945 (incorporated herein by reference), one such suitable means for obtaining gene fragments in accordance with the invention, e.g., those corresponding to the A domain of ACE (ACE 40, aa 32-367) and subdomain of ACE 40, namely ACE19 (aa 174-319), is to use a process wherein they are amplified by using PCR and chromosomal DNA from Enterococcus faecalis strain EF1 as template. In this process, the resulting gene fragments were subcloned in E. coli expression vector pQE-30 and transformed into E. coli strain JM101. Recombinant proteins with His tag at the N terminus were produced by inoculating 1-liter cultures of Luria broth, containing 100 micrograms/ml ampicillin, with 40 ml of overnight cultures of the expression constructs. Following 2.5 h of growth at 370C, the cells were induced with 0.2 mM isopropyl-1-beta-D-thiogalactoside (IPGT) for another 3 h. Bacteria were harvested by centrifugation, the supernatant decanted and the cell paste frozen at −80° C. Cells were later thawed at 22° C., suspended in PBS and lysed using a French press. Insoluble cell debris was removed by centrifugation at 30,000×g for 30 min, followed by filtration through a 0.45-micrometer membrane. Supernatant was applied to a 5-mi Ni²⁺ charged HiTrap chelating column (Pharmacia) and bound protein eluted with a 200 ml linear gradient of 0-200 mM imidazole in 4 mM Tris-HCl, 100 mM NaCl, pH 8.0. Fractions corresponding to the recombinant ACE40 or ACE19, were pooled and dialyzed against 25 MM Tris-HCl, pH 8.0. Dialyzed protein was passed over a 5-ml HiTrap Q column (Phamacia) and bound protein eluted with a 200 ml linear gradient of 0-0.5 M NaCl in 25 mM Tris-HCl, pH 8.0. From these obtained recombinant proteins, generation of the monoclonal antibodies in accordance with the invention may thus proceed in any of a number of conventional methods well known in the art as described further below.

In another suitable example, the recombinant proteins and peptides of the invention may also be prepared again using E. coli vector pQE-30 as an expression vector. In this example, using PCR, the A domain of ACE, namely ACE40 or amino acids 32-367 of the ACE protein from E. faecalis was amplified and subcloned into the E. coli expression vector PQE-30 (Qiagen), which allows for the expression of a recombinant fusion protein containing six histidine residues. This vector was subsequently transformed into the E. coli strain ATCC 55151, grown in a 15-liter fermentor to an optical density (OD₆₀₀) of 0.7 and induced with 0.2 mM isopropyl-1-beta-D galactoside (IPTG) for 4 hours. The cells were harvested using an AG Technologies hollow-fiber assembly (pore size of 0.45 μm) and the cell paste frozen at −80° C. Cells were lysed in 1×PBS (10 mL of buffer/1 g of cell paste) using 2 passes through the French Press @ 1100 psi. Lysed cells were spun down at 17,000 rpm for 30 minutes to remove cell debris. Supernatant was passed over a 5-mL HiTrap Chelating (Pharmacia) column charged with 0.1M NiCl₂. After loading, the column was washed with 5 column volumes of 10 mM Tris, pH 8.0, 100 mM NaCl (Buffer A). Protein was eluted using a 0-100% gradient of 10 mM Tris, pH 8.0, 100 mM NaCl, 200 mM imidazole (Buffer B) over 30 column volumes and useful fractions were dialyzed in lx PBS.

The protein was then put through an endotoxin removal protocol. Buffers used during this protocol were made endotoxin free by passing over a 5-mL Mono-Q sepharose (Pharmacia) column. Protein was divided evenly between 4×15 mL tubes. The volume of each tube was brought to 9 mL with Buffer A. 1 mL of 10% Triton X-114 was added to each tube and incubated with rotation for 1 hour at 4° C. Tubes were placed in a 37° C. water bath to separate phases. Tubes were spun down at 2,000 rpm for 10 minutes and the upper aqueous phase from each tube was collected and the detergent extraction repeated. Aqueous phases from the 2nd extraction were combined and passed over a 5-mL IDA chelating (Sigma) column, charged with 0.1M NiCl₂ to remove remaining detergent. The column was washed with 9 column volumes of Buffer A before the protein was eluted with 3 column volumes of Buffer B. The eluant was passed over a 5-mL Detoxigel (Sigma) column and the flow-through collected and reapplied to the column. The flow-through from the second pass was collected and dialyzed in 1×PBS. The purified product was analyzed for concentration, purity and endotoxin level before administration into the mice.

The amino acid sequence for ACE40 obtained in this manner is shown herein as amino acids 32-567 in SEQ ID NO:1, and is encoded by nucleic acids at the corresponding location by the sequence in SEQ ID NO:2, namely nucleotides 94-1701, or degenerates thereof.

In accordance with the invention, following isolation of the target protein or peptide, monoclonal antibodies can be produced by a number of suitable ways. For example, in one preferred method, these proteins are used to generate a panel of murine monoclonal antibodies. In one suitable method, monoclonal antibodies against ACE 40 and ACE19 were produce essentially as described by Kohler and Milstein with minor modifications. Balb/c mice were injected intraperitoneally five times at 10 days intervals with 50 micrograms of each recombinant protein. The antigen was emulsified with an equal volume of complete Freund's adjuvant for the first immunization, followed by three injections in incomplete adjuvant. The mice were bled, and the sera were tested for reactivity to the purified ACE 40 or ACE19 using ELISA and Western blot. For the final immunization, the antigen was given in saline. Three days later, the lymphocytes were isolated from spleen and fused to a Sp2/0 Ag.14 mouse myeloma cell line (ATCC#1581) at a ratio of 5:1 using 50% polyethylene glycol 4000. The suspended cells were first grown and selected in high glucose Dulbecco's modified Eagle's medium/RPMI 1640 (1:1) medium (Sigma) containing 2% hypoxantine/aminopterin/thymidine (Sigma), 25 glutamine, 2% penicillin, and 2% streptomycin and supplemented with 10% (v/v) foetal bovine serum. After a week, the hypoxantine/aminopterin/thymidine medium was progressively replaced by culturing cloned hybridomas in a medium consisting of Dulbecco's modified Eagle's medium/RPMI 1640 supplemented with 10% (v/v) foetal bovine serum. After stable clones were generated, hybridoma were grown in a serum-free medium made of Dulbecco's modified Eagle's medium/RPMI 1640 containing 1% (v/v) Nutridoma-SR (Roche Molecular Biochemicals, Mannheim, Germany) and antibiotics. Supernatants of the cell cultures were screened by ELISA on day 10, and hybridomas positive for the antibodies against ACE 40 or ACE19 were subcultured to a density of 1 cell per well by limiting dilution and further characterized by ELISA and Western blot. Thirty and six positive clones were obtained against ACE40 and ACE19, respectively.

In another suitable method, a group of suitable mice, such as Balb/C mice, received a series of subcutaneous immunizations of the target protein in solution or mixed with an appropriate adjuvant. Three days after the final delivery of adjuvant, the spleens were removed, teased into a single cell suspension and the lymphocytes harvested. The lymphocytes were then fused to a SP2/0-Ag14 myeloma cell line (ATCC #1581). Cell fusion, subsequent plating and feeding were performed according to the Production of Monoclonal Antibodies protocol from Current Protocols in Immunology (Chapter 2, Unit 2.).

Any clones that were generated from the fusion were then screened for specific antibody production using a standard ELISA assay. Positive clones were expanded and tested further. Fifteen positive clones were originally identified and cloned by limiting dilution for further characterization. Single cell clones were tested for activity in a direct binding ELISA, a modified ELISA to measure inhibition of collagen binding, whole bacterial cell binding by flow cytometry and affinity for peptide binding by Biacore analysis.

Although production of antibodies using recombinant forms of the peptides described above is preferred, antibodies may be generated from the natural isolated and purified ACE or CNA peptides as well, and monoclonal antibodies can be generated in the same manner as described above.

In addition, it is possible to obtain polyclonal antibodies in accordance with the invention which have cross-reactive properties. For example, certain polyclonal mouse sera from mice immunized with ACE40 appear to have cross-reactivity with several strains of E. faecalis and one strain of E. faecium, as has been shown in flow cytometry.

As shown in data below, immunizations to generate monoclonal antibodies in accordance with the present invention directed to ACE40, ACE19 and CNA19 have yielded monoclonal antibodies which have shown to be cross-reactive. Specific monoclonal antibodies generated in this matter are described further below.

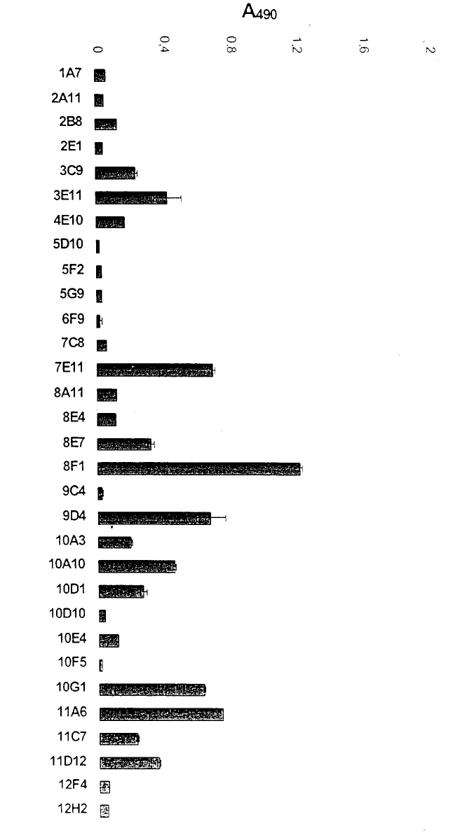

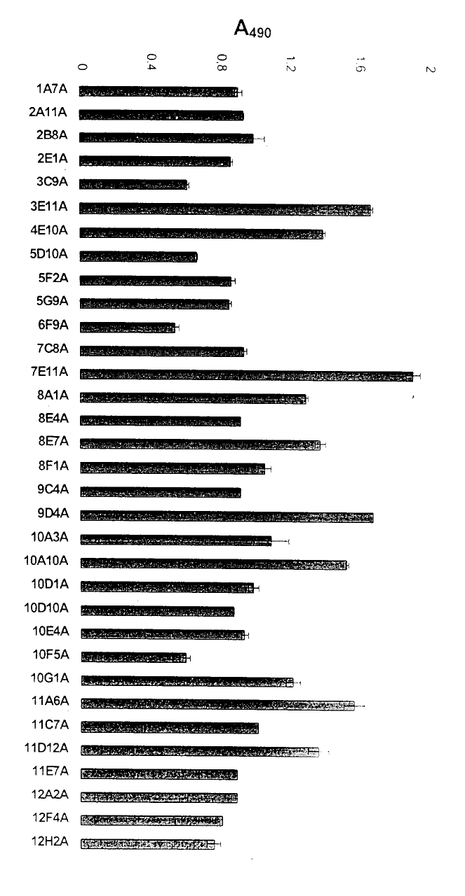

In accordance with the invention, MAbs generated against ACE40 (aa 32-367) or CNA19 were also tested for reactivity with cells of Enterococcus faecium or Streptococcus pyogenes adhering to immobilized collagen. As expected, all the mAbs against ACE40 were observed to bind to the surface of E. faecalis, suggesting that ACE40 contains epitopes that are maintained on the antigen expressed on the bacterial surface (as shown in FIG. 3). In addition, many anti-ACE40 mAbs in accordance with the invention (e.g., 7E11, 8F1, 9D4, 10G1 and 11A6) were observed to be cross-reactive and bound to the immobilized cells of Enterococcus faecium, a separate Enterococcus bacterial species (see FIG. 1).

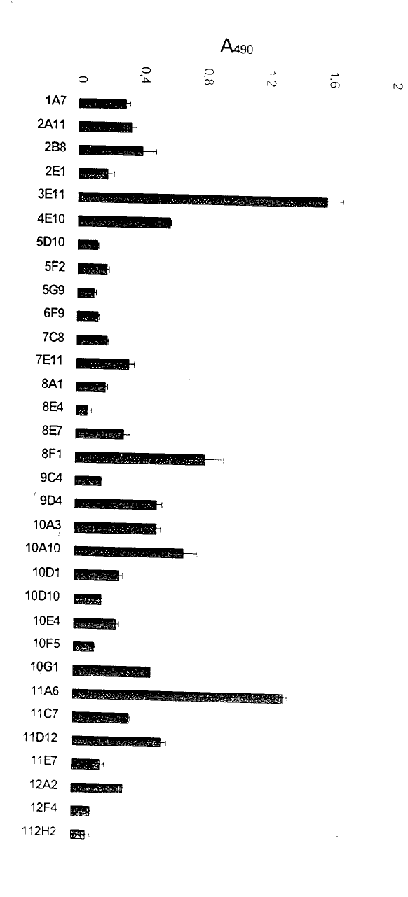

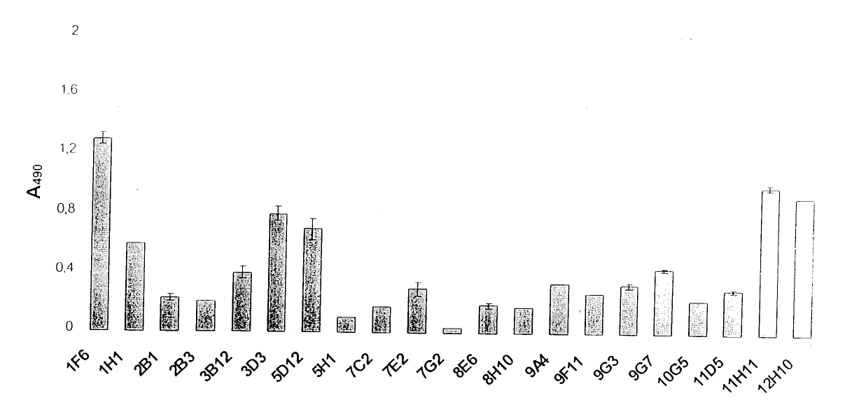

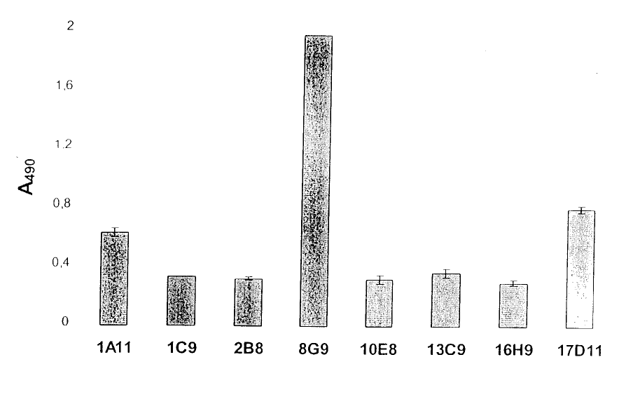

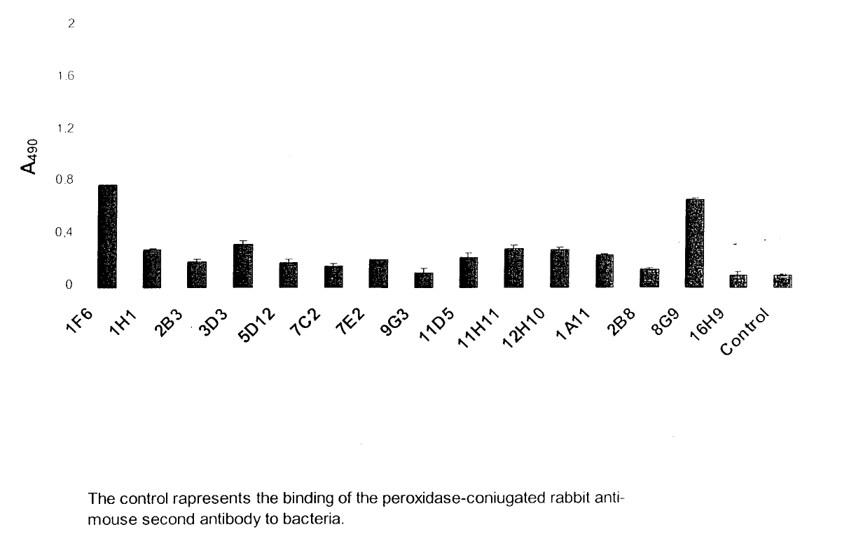

A similar screening performed incubating a panel of mAbs against CNA19 or CNA55 (aa 30-529) with E. faecium cells adhering to collagen also revealed mAbs (e.g., 1F6, 3D3, 11H11, 12H10 and 8G9) which also show cross reactivity with other bacteria (FIGS. 5 and 6). A screening conducted assaying the cross reactivity of mAbs against ACE40 with Streptococcus pyogenes cells adhering to immobilized collagen revealed that the mAbs of the invention (e.g., 3E11, 8F1, 10A10 and 11A6) also showed cross-reactivity against S. pyogenes (see FIG. 4). It is noteworthy that these mAbs show a similar reactivity with immunodeterminants expressed on the surface of E. faecium. MAbs against CNA also showed some cross reactivity with cells of S. pyogenes, and certain mAbs (1F6 and 8G9) bound to the bacteria at levels significantly higher than those of the controls (FIG. 7).

In accordance with the present invention, the cross-reactive monoclonal antibodies of the invention may be utilized in many therapeutic and other useful applications as set forth below.

Pharmaceutical Compositions

As would be recognized by one skilled in the art, the antibodies of the present invention may also be formed into suitable pharmaceutical compositions for administration to a human or animal patient in order to treat or prevent an infection caused by staphylococcal bacteria. Pharmaceutical compositions containing the antibodies of the present invention, or effective fragments thereof, may be formulated in combination with any suitable pharmaceutical vehicle, excipient or carrier that would commonly be used in this art, including such as saline, dextrose, water, glycerol, ethanol, other therapeutic compounds, and combinations thereof. As one skilled in this art would recognize, the particular vehicle, excipient or carrier used will vary depending on the patient and the patient's condition, and a variety of modes of administration would be suitable for the compositions of the invention, as would be recognized by one of ordinary skill in this art. Suitable methods of administration of any pharmaceutical composition disclosed in this application include, but are not limited to, topical, oral, anal, vaginal, intravenous, intraperitoneal, intramuscular, subcutaneous, intranasal and intradermal administration.

For topical administration, the composition is formulated in the form of an ointment, cream, gel, lotion, drops (such as eye drops and ear drops), or solution (such as mouthwash). Wound or surgical dressings, sutures and aerosols may be impregnated with the composition. The composition may contain conventional additives, such as preservatives, solvents to promote penetration, and emollients. Topical formulations may also contain conventional carriers such as cream or ointment bases, ethanol, or oleyl alcohol.

Additional forms of antibody compositions, and other information concerning compositions, methods and applications with regard to other MSCRAMM® proteins and MSCRAMM® peptides will generally also be applicable to the present invention involving monoclonal antibodies and are disclosed, for example, in U.S. Pat. No. 6,288,214 (Hook et al.), incorporated herein by reference.

The antibody compositions of the present invention which are generated in particular against the ACE19, ACE40 and CNA19 peptides as set forth above may also be administered with a suitable adjuvant in an amount effective to enhance the immunogenic response against the conjugate. For example, suitable adjuvants may include alum (aluminum phosphate or aluminum hydroxide), which is used widely in humans, and other adjuvants such as saponin and its purified component Quil A, Freund's complete adjuvant, RIBI adjuvant, and other adjuvants used in research and veterinary applications. Still other chemically defined preparations such as muramyl dipeptide, monophosphoryl lipid A, phospholipid conjugates such as those described by Goodman-Snitkoff et al. J. Immunol. 147:410-415 (1991) and incorporated by reference herein, encapsulation of the conjugate within a proteoliposome as described by Miller et al., J. Exp. Med. 176:1739-1744 (1992) and incorporated by reference herein, and encapsulation of the protein in lipid vesicles such as Novasome™ lipid vesicles (Micro Vescular Systems, Inc., Nashua, N.H.) may also be useful.

In any event, the antibody compositions of the present invention will thus be useful for interfering with, modulating, inhibiting binding interactions involving collagen and collagen binding proteins as would take place with bacteria from staphylococcal, streptococcal and enterococcal species. Accordingly, the present invention will have particular applicability in developing compositions and methods of preventing or treating staphylococcal infection, and in inhibiting binding of staphylococcal bacteria to host tissue and/or cells.

Treating or Protecting Against Infections

In accordance with the present invention, methods are provided for preventing or treating a bacterial infection which comprise administering an effective amount of the monoclonal antibodies as described above in amounts effective to treat or prevent the infection. In addition, these monoclonal antibodies will be particularly useful in impairing the binding of a variety of bacteria to collagen, and have thus proved effective in treating or preventing infection from bacteria such as staphylococcus, streptococcus or enterococcus. The antibodies in accordance with the invention are particularly effective in that they have been shown to be cross-reactive across a variety of bacterial species and will thus improve the effectiveness and efficiency of compositions based on the monoclonals of the present invention.

Accordingly, in accordance with the invention, administration of the antibodies of the present invention in any of the conventional ways described above (e.g., topical, parenteral, intramuscular, etc.), and will thus provide an extremely useful method of treating or preventing bacterial infections in human or animal patients. By effective amount is meant that level of use, such as of an antibody titer, that will be sufficient to either prevent adherence of the bacteria, to inhibit binding of bacteria to host cells and thus be useful in the treatment or prevention of a bacterial infection. As would be recognized by one of ordinary skill in this art, the level of antibody titer needed to be effective in treating or preventing infections will vary depending on the nature and condition of the patient, and/or the severity of the pre-existing infection.

Vaccines and Humanized Antibodies

The isolated antibodies of the present invention, or active fragments thereof, may also be utilized in the development of vaccines for passive immunization against bacterial infections. Further, when administered as pharmaceutical composition to a wound or used to coat medical devices or polymeric biomaterials in vitro and in vivo, the antibodies of the present invention, may be useful in those cases where there is a previous infection because of the ability of these antibodies to further restrict and inhibit bacterial binding to collagen and thus limit the extent and spread of the infection. In addition, the antibody may be modified as necessary so that, in certain instances, it is less immunogenic in the patient to whom it is administered. For example, if the patient is a human, the antibody may be “humanized” by transplanting the complimentarity determining regions of the hybridoma-derived antibody into a human monoclonal antibody as described, e.g., by Jones et al., Nature 321:522-525 (1986) or Tempest et al. Biotechnology 9:266-273 (1991) or “veneered” by changing the surface exposed murine framework residues in the immunoglobulin variable regions to mimic a homologous human framework counterpart as described, e.g., by Padlan, Molecular Imm. 28:489-498 (1991), these references incorporated herein by reference. Even further, when so desired, the monoclonal antibodies of the present invention may be administered in conjunction with a suitable antibiotic to further enhance the ability of the present compositions to fight bacterial infections.

In a preferred embodiment, the antibodies may also be used as a passive vaccine which will be useful in providing suitable antibodies to treat or prevent a bacterial infection. As would be recognized by one skilled in this art, a vaccine may be packaged for administration in a number of suitable ways, such as by parenteral (i.e., intramuscular, intradermal or subcutaneous) administration or nasopharyngeal (i.e., intranasal) administration. One such mode is where the vaccine is injected intramuscularly, e.g., into the deltoid muscle, however, the particular mode of administration will depend on the nature of the bacterial infection to be dealt with and the condition of the patient. The vaccine is preferably combined with a pharmaceutically acceptable carrier to facilitate administration, and the carrier is usually water or a buffered saline, with or without a preservative. The vaccine may be lyophilized for resuspension at the time of administration or in solution.

The preferred dose for administration of an antibody composition in accordance with the present invention is that amount will be effective in preventing of treating a bacterial infection, and one would readily recognize that this amount will vary greatly depending on the nature of the infection and the condition of a patient. As indicated above, an “effective amount” of antibody or pharmaceutical agent to be used in accordance with the invention is intended to mean a nontoxic but sufficient amount of the agent, such that the desired prophylactic or therapeutic effect is produced. Accordingly, the exact amount of the antibody or a particular agent that is required will vary from subject to subject, depending on the species, age, and general condition of the subject, the severity of the condition being treated, the particular carrier or adjuvant being used and its mode of administration, and the like. Accordingly, the “effective amount” of any particular antibody composition will vary based on the particular circumstances, and an appropriate effective amount may be determined in each case of application by one of ordinary skill in the art using only routine experimentation. The dose should be adjusted to suit the individual to whom the composition is administered and will vary with age, weight and metabolism of the individual. The compositions may additionally contain stabilizers or pharmaceutically acceptable preservatives, such as thimerosal (ethyl(2-mercaptobenzoate-S)mercury sodium salt) (Sigma Chemical Company, St. Louis, Mo.).

Coating Devices

Medical devices or polymeric biomaterials to be coated with the monoclonal antibodies and/or compositions described herein include, but are not limited to, staples, sutures, replacement heart valves, cardiac assist devices, hard and soft contact lenses, intraocular lens implants (anterior chamber or posterior chamber), other implants such as corneal inlays, kerato-prostheses, vascular stents, epikeratophalia devices, glaucoma shunts, retinal staples, scleral buckles, dental prostheses, thyroplastic devices, laryngoplastic devices, vascular grafts, soft and hard tissue prostheses including, but not limited to, pumps, electrical devices including stimulators and recorders, auditory prostheses, pacemakers, artificial larynx, dental implants, mammary implants, penile implants, cranio/facial tendons, artificial joints, tendons, ligaments, menisci, and disks, artificial bones, artificial organs including artificial pancreas, artificial hearts, artificial limbs, and heart valves; stents, wires, guide wires, intravenous and central venous catheters, laser and balloon angioplasty devices, vascular and heart devices (tubes, catheters, balloons), ventricular assists, blood dialysis components, blood oxygenators, urethral/ureteral/urinary devices (Foley catheters, stents, tubes and balloons), airway catheters (endotracheal and tracheostomy tubes and cuffs), enteral feeding tubes (including nasogastric, intragastric and jejunal tubes), wound drainage tubes, tubes used to drain the body cavities such as the pleural, peritoneal, cranial, and pericardial cavities, blood bags, test tubes, blood collection tubes, vacutainers, syringes, needles, pipettes, pipette tips, and blood tubing.

It will be understood by those skilled in the art that the term “coated” or “coating”, as used herein, means to apply the antibody or active fragment, or pharmaceutical composition derived therefrom, to a surface of the device, preferably an outer surface that would be exposed to a bacterial infection. The surface of the device need not be entirely covered by the protein, antibody or active fragment.

As indicated above, the monoclonal antibodies of the present invention, or active portions or fragments thereof, are particularly useful for interfering with the initial physical interaction between a bacterial pathogen responsible for infection and a mammalian host, such as the adhesion of the bacteria to mammalian extracellular matrix proteins such as collagen, and this interference with the physical interaction may be useful both in treating patients and in preventing or reducing bacteria infection on in-dwelling medical devices to make them safer for use. Still other applications include suitable diagnostic kits for determining the presence of bacteria or proteins that will bind to the monoclonal antibodies of the invention. These diagnostic kits will include the antibodies of the invention along with suitable means for detecting binding by that antibody such as would be readily understood by one skilled in this art. For example, the means for detecting binding of the antibody may comprise a detectable label that is linked to said antibody.

In short, the monoclonal antibodies of the present invention as described above recognize epitopes on a variety of bacterial species and are thus cross-reactive and extremely useful in treating or preventing bacterial infections in human and animal patients and in a variety of other applications including use on medical or other in-dwelling devices.

### EXAMPLES

The following examples are provided which exemplify aspects of the preferred embodiments of the present invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Expression and Purification of Recombinant ACE40 and ACE19

As described, for example, in J. Biol. Chem. 1999, 274, 26939-26945 (incorporated herein by reference), gene fragments corresponding to the A domain of ACE (ACE 40, aa 32-367) and subdomain of ACE 40, namely ACE19 (aa 174-319), can be obtained in a process wherein they are amplified by using PCR and chromosomal DNA from Enterococcus faecalis strain EF1 as template. The resulting gene fragments were subcloned in E. coli expression vector pQE-30 and transformed into E. coli strain JM101. Recombinant proteins with His tag at the N terminus were produced by inoculating 1-liter cultures of Luria broth, containing 100 micrograms/ml ampicillin, with 40 ml of overnight cultures of the expression constructs. Following 2.5 h of growth at 37° C., the cells were induced with 0.2 mM isopropyl-1-beta-D-thiogalactoside (IPGT) for another 3 h. Bacteria were harvested by centrifugation, the supernatant decanted and the cell paste frozen at −80° C. Cells were later thawed at 22° C., suspended in PBS and lysed using a French press. Insoluble cell debris was removed by centrifugation at 30,000×g for 30 min, followed by filtration through a 0.45-micrometer membrane. Supernatant was applied to a 5-ml Ni²⁺ charged HiTrap chelating column (Pharmacia) and bound protein eluted with a 200 ml linear gradient of 0-200 mM imidazole in 4 mM Tris-HCl, 100 mM NaCl, pH 8.0. Fractions corresponding to the recombinant ACE40 or ACE19, were pooled and dialyzed against 25 MM Tris-HCl, pH 8.0. Dialyzed protein was passed over a 5-ml HiTrap Q column (Phamacia) and bound protein eluted with a 200 ml linear gradient of 0-0.5 M NaCl in 25 mM Tris-HCl, pH 8.0.

### Example 2

### Generation of Monoclonal Antibodies

Monoclonal antibodies against ACE 40 and ACE19 were produced essentially as described by Kohler and Milstein with minor modifications. Balb/c mice were injected intraperitoneally five times at 10 days intervals with 50 micrograms of each recombinant protein. The antigen was emulsified with an equal volume of complete Freund's adjuvant for the first immunization, followed by three injections in incomplete adjuvant. The mice were bled, and the sera were tested for reactivity to the purified ACE 40 or ACE19 using ELISA and Western blot. For the final immunization, the antigen was given in saline. Three days later, the lymphocytes were isolated from spleen and fused to a Sp2/0 Ag.14 mouse myeloma cell line (ATCC#1581) at a ratio of 5:1 using 50% polyethylene glycol 4000. The suspended cells were first grown and selected in high glucose Dulbecco's modified Eagle's medium/RPMI 1640 (1:1) medium (Sigma) containing 2% hypoxantine/aminopterin/thymidine (Sigma), 25 glutamine, 2% penicillin, and 2% streptomycin and supplemented with 10% (v/v) foetal bovine serum. After a week, the hypoxantine/aminopterin/thymidine medium was progressively replaced by culturing cloned hybridomas in a medium consisting of Dulbecco's modified Eagle's medium/RPMI 1640 supplemented with 10% (v/v) foetal bovine serum. After stable clones were generated, hybridoma were grown in a serum-free medium made of Dulbecco's modified Eagle's medium/RPMI 1640 containing 1% (v/v) Nutridoma-SR (Roche Molecular Biochemicals, Mannheim, Germany) and antibiotics. Supernatants of the cell cultures were screened by ELISA on day 10, and hybridomas positive for the antibodies against ACE 40 or ACE19 were subcultured to a density of 1 cell per well by limiting dilution and further characterized by ELISA and Western blot. Thirty and six positive clones were obtained against ACE40 and ACE19, respectively.

### Example 3

### Binding of Anti-ACE 40 (aa 32-367) Monoclonal Antibodies to Enterococcus faecium, Strain 935/01, Adhering to Immobilized Collagen

The following examples reflect tests done concerning the immunological cross reactivity of monoclonal antibodies in accordance with the present invention including those directed against recombinant collagen-binding fragments CNA19 (aa 151-318) from S. aureus, as well as those generated against ACE40 (aa 32-367) and ACE19 (174-319) from E. faecalis.

The following tests were done as reflected in the drawing figures:

Tests as Shown in FIG. 1

Monoclonal antibodies to ACE40 were generated as described above using Enterococcus faecium, strain 935/01, and these antibodies were tested for their ability to recognize E. faecium as determined by their adherence to immobilized collagen. In these tests, microtiter wells were coated with 100 microliters of 50 mM sodium carbonate, pH 9.5, containing 10 micrograms of collagen type II per ml. Additional protein binding sites in the wells were blocked by incubation for 1 h with 200 microliters of 0.2% (wt/vol) bovine serum albumin (BSA) in 10 mM sodium phosphate, pH 7.4, containing 0.13 M NaCl (PBS). The wells were then washed five times with PBST (0.1% Tween 20 in PBS) and incubated with 2×108 cells of Enterococcus faecium, strain 935/01, for 2 h at 37° C. After washing (×3) with PBS, wells containing collagen-bound bacteria were incubated with 2 micrograms of each mAb for 2 h at 37° C. The wells were subsequently washed with PBS and antibody associated with the wells was detected by incubation for 1 h at 22° C. with peroxidase-conjugated rabbit anti-mouse IgG diluted 1:1000. After being washed the conjugated enzyme was reacted with o-phenylenediamine dihydrochloride and the absorbance at 492 nm was monitored with a microplate reader. Values represent the means ±SD of duplicate wells. FIG. 1 thus shown the binding of anti-ACE40 monoclonal antibodies to Enterococcus faecium strain 935/01, as adhering to immobilized collagen.

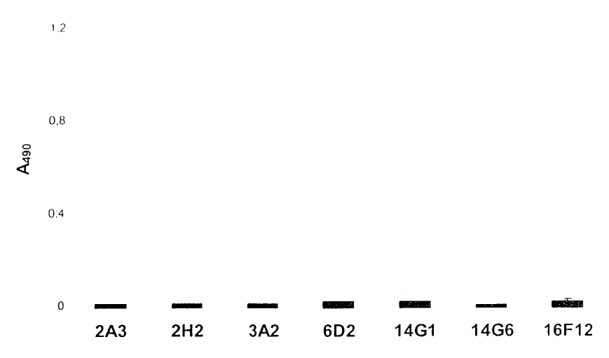

Tests as Shown in FIG. 2

This figure shows the binding of anti-ACE 19 (aa 174-319) monoclonal antibodies to E. faecium, strain 935/01, adhering to immobilized collagen, wherein attachment of enterococci to collagen, binding and detection of mAbs to collagen bound bacteria were performed as reported in FIG. 1.

Tests as Shown in FIG. 3

This figure shows the binding of anti-ACE 40 monoclonal antibodies to Enterococcus faecalis, strain 687097, adhering to immobilized collagen. Attachment of enterococcal cells to collagen and binding and detection of anti-ACE 40 mAbs to bacteria adhering to immobilized collagen were performed as reported in FIG. 1.

Tests as Shown in FIG. 4

This figure shows the binding of anti-ACE mAbs to cells of Streptococcus pyogenes 64/14 adhering to immobilized collagen. Microtiter wells were coated with 100 microliters of 50 mM sodium carbonate, pH 9.5, containing 10 micrograms of collagen type 11 per ml. Additional protein binding sites in the wells were blocked by incubation for 1 h with 200 microliters of 0.2% (wt/vol) bovine serum albumin (BSA) in PBS. The wells were then washed five times with PBST (0.1% Tween 20 in PBS) and incubated with 3×10⁸ cells of Streptococcus pyogenes, strain 64/14, for 2 h at 37° C. After washing (x 3) with PBS, collagen-bound bacteria were overlaid with 10 micrograms of human IgG and incubated for 60 min at 22° C. After extensive washing, collagen-bound bacteria were assayed for anti-ACE antibody binding as reported in FIG. 1.

Tests as Shown in FIG. 5

This figure shows the binding of anti-CNA19 (aa 151-318) to cells of Enterococcus faecium 935/01 adhering to immobilized collagen. Attachment of enterococcal cells to collagen coated wells and binding assays of anti-CNA 19 mAbs to bacteria were performed as reported in FIG. 1.

Tests as Shown in FIG. 6

This figure shows the binding of anti-CNA55 (aa 30-529) to cells of Enterococcus faecium 935/01 adhering to immobilized collagen. Attachment of enterococcal cells to collagen coated wells and binding assays of anti-CNA 55 mAbs to bacteria were performed as reported in FIG. 1.

Tests as Shown in FIG. 7

This figure shows the binding of a selected number of mAbs against CNA19 and CNA55 to cells of Streptococcus pyogenes 64/14 adhering to immobilized collagen. Attachment of streptococcal cells to collagen coated wells and binding assays of anti-CNA mAbs to bacteria were performed as reported in FIG. 4.

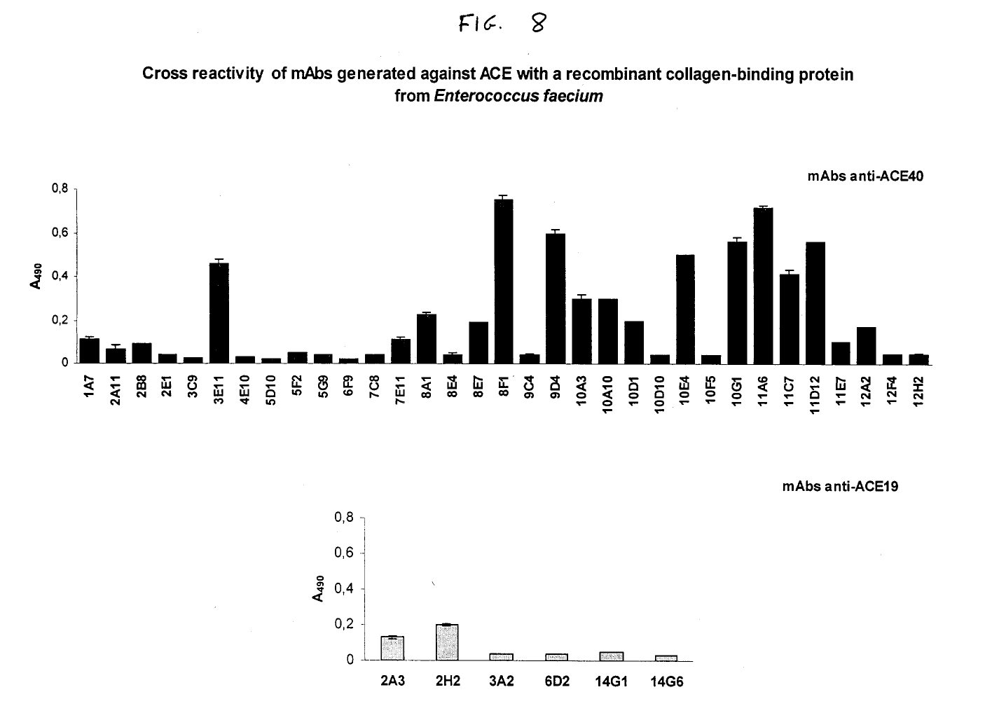

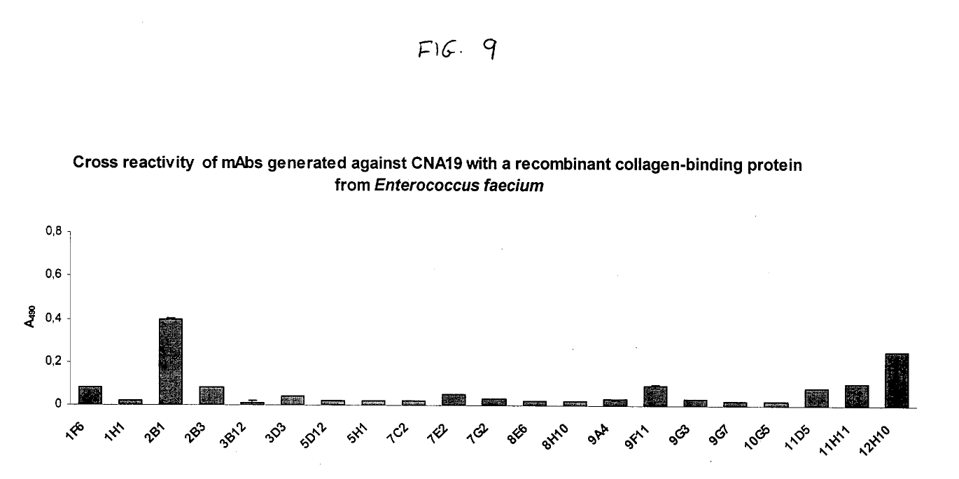

Tests as Shown in FIGS. 8-9

These figures show the binding of monoclonals against ACE and CNAN to a collagen-binding protein from E. faecium identified as Acm. The reactivity of mAbs against ACE and CNA for Acm was observed in that some anti-ACE mAbs recognize epitopes in Acm. These data fit well with the previous finding where anti-ACE40 mAbs reacted with cells of E. faecium attached to collagen coated wells.

Results and Discussion

The present tests evidence the immunological cross reactivity of monoclonal antibodies directed against recombinant collagen-binding fragments CNA19 (aa 151-318) from S. aureus, along with the peptides ACE19 (aa 174-319) and ACE40 (aa 32-367) from E. faecalis. mAbs generated against ACE40 (aa 32-367) or CNA19 were tested for reactivity with cells of Enterococcus faecium or Streptococcus pyogenes adhering to immobilized collagen. As expected, all the mAbs against ACE40 bound to the surface of E. faecalis, suggesting that ACE40 contains epitopes that are maintained on the antigen expressed on the bacterial surface (FIG. 3). An adequate number of anti-ACE40 mAbs (7E11, 8F1, 9D4, 10G1 and 11A6) bound to the immobilized cells of Enterococcus faecium, a bacterial species strictly related to E. faecalis (FIG. 1). This finding indicates that specific epitopes are shared between ACE of E. faecalis and MSCRAMM® proteins on the surface of E. faecium. Inasmuch as the strain of E. faecium used in this study is a positive collagen binder, it is plausible that the epitopes recognized by anti-ACE 40 mAbs are located within a collagen-binding MSCRAMM® protein.

A similar screening performed incubating a panel of mAbs against CNA19 or CNA55 (aa 30-529) with E. faecium cells adhering to collagen revealed that a few mAbs (1F6, 3D3, 11H11, 12H10 and 8G9) show a moderate cross reactivity with bacteria (FIGS. 5 and 6).

A screening conducted assaying the cross reactivity of mAbs against ACE40 with Streptococcus pyogenes cells adhering to immobilized collagen revealed the mAbs 3E11, 8F1, 10 A10 and 11A6 gave the most prominent signal (FIG. 4). It is noteworthy that these mAbs show a similar reactivity with immunodeterminants expressed on the surface of E. faecium. Therefore, it is plausible that surface components of S. pyogenes and E. faecium share epitopes recognized by these mAbs.

MAbs against CNA showed a diffuse weak cross reactivity with cells of S. pyogenes, except mAbs 1 F6 and 8G9 that bound to the bacteria at levels significantly higher than those of the controls (FIG. 7). The cross reactivity of 8G9 with S. pyogenes cells is reminiscent of signal response detected incubating 8G9 with E. faecium cells adhering to immobilized collagen. Thus, we meet another case of molecular mimicry where epitopes of CNA of S. aureus are shared with antigenic determinants of S. pyogenes and E. faecium.

The following references are incorporated herein by reference as if set forth in the specification in their entirety:

1. Langermann, S., S. Palaszynski, M. Barnhart, G. Auguste, J. S. Pinker, J. Burlein, P. Barren, S. Koening, S. Leath, C. H. Jones, and S. J. Hutgren. 1997. Prevention of mucosal Escheriachia coli infections by FimH-adhesin-based systemic vaccination. Science. 276: 607-611

2. Sirakowa, T., P. E. Kolattukudy, D. Marwin, J. Billy, E. Leake, D. Lim, T. Demaria, and L. Bakalets. 1994. Role of fimbriae expressed by non typeable Haemophilus influenzae in pathogenesis of a protection otitis media and relatedness of the fimbrine subunit to outer membrane protein A. Infect. Immun. 62: 2002-2020

3. Hanson, M. S., D. R. Cassatt, B. P. Guo, N. K. Patel, M. P. McCarthy, D. W. Dorward, and M. Hook. 1998. Active and passive immunity against Borrelia burgdorferi decorin-binding protein A (DpbA) protects against infection. Infect. Immun. 66: 2143-2153

4. Patti, J. M., J. O. Boles, and M. Hook. 1993. Identification and biochemical characterization of ligand binding domain of the collagen adhesin from Staphylococcus aureus. Biochemistry. 32: 11428-11435

5. Rich, R. L., B. Demler, K. Ashby, C. C. S. Deivanayagam, J. W. Petrich, J. M. Patti., S. V. L. Narayana, and M. Hook. 1998. Domain structure of the Staphylococcus aureus adhesin. Biochemistry. 37: 15423-15433

6. Symersky, J., J. M. Patti, M. Carson, K. House-Pompeo, M. Teale, D. Moore, L. Jin, A. Schneider, L. J. DeLucas, M. Hook, and S. V. L. Narayana. 1997. Structure of the collagen-binding domain from a Staphylococcus aureus adhesin. Nature Struct. Biol. 4: 833-838

7. Switalski, L. M., J. M. Patti, W. Butcher, A. G. Gristina, P. Speziale, and M. Hook. 1993. A collagen receptor on Staphylococcus aureus strain isolated from patients with septic arthritis mediates adhesion to cartilage. Mol. Microbiol. 7: 99-107.

8. Patti, J. M., T. Bremell, D. Krajewska-Pietrasik, A. Abdelnour, A. Tarkowski, C. Rydèn, and M. Hook. 1994. The Staphylococcus aureus collagen adhesin is a virulence determinant in experimental septic arthritis. Infect. Immun. 62: 152-161

9. Nilsson, I-M., J. M. Patti, T. Bremell, M. Hook, and A. Tarkowski. 1998. Vaccination with a recombinant fragment of collagen adhesin provides protection against Staphylococcus aureus-mediated septic death. 101: 2640-2649

10. Visai, L., Y. Xu, F. Casolini, S. Rindi, M. Hook, and P. Speziale. 2000. Monoclonal antibodies to CNA, a collagen-binding microbial surface component recognizing adhesive matrix molecules, detach Staphylococcus aureus from a collagen substrate

11. Xiao, J., M. Hook, G. M. Weinstock, and B. E. Murray. 1998. Conditional adherence of Enterococcus faecalis to extracellular matrix proteins. FEMS Immunol. Med. Microbiol. 21. 287-295

12. Rich, R. L., B. Kreikemeyer, R. T. Owens, S. LaBrenz, S. V. L. Narayana, G. M. Weinstock, B. E. Murray, and M. Hook. 1999. Ace is a collagen-binding MSCRAMM from Enterococcus faecalis. J. Biol. Chem. 274: 26939-26945

13. Zhang. H., I. Kour, D. W. Nielsel, G. S. Seetharamaiah, J. W. Peterson, and G. R. Klimpel. 1997. Lipoprotein from Yersinia enterocolitica contains epitopes that cross react with human thyrotropin receptor. 158: 1976-1983

14. Hernandez-Munain. C., J. L. De-Diego, P. Bonay, N. Girones, and M. Fresno. 1993. GP50/55, a membrane antigen of Trypanosoma cruzi involved in autoimmunity and immunosuppression. Biol. Res. 26: 209-218

15. Manjula, B. N., B. L. Trus, and V. A. Fischetti. 1985. Presence of two distinct regions in the coiled-coil structure of the streptococcal Pep M 5 protein: relationship to mammalian coiled-coil proteins and implications to its biological properties. Proc. Natl. Acad. Sci. USA 82: 1064-1068

16. Quinn, A., S. Kosanke, V. A. Fischetti, S. M. Factor, and M. W. Cunningham. 2001. Induction of autoimmune valvular heart disease by recombinant streptococcal M protein. Infect. Immun. 69: 4072-4078

17. Fontàn. P. A., V. Pancholi, M. M. Nociari, and V. A. Fischetti. 2000. Antibodies to streptococcal surface enolase react with human alpha enolase: implications in poststreptococcal sequelae. J. Infect. Dis. 182: 1712-1721

18. Prehm. S., C. Herrington, V. Volker, N I. Briko, E I. Blinnikova, A. Schiedel, and P. Prehm. 1995. Antibodies against proteins of streptococcal hyaluronate sybthase bind to human fibroblasts are present in patients with rheumatic fever. J. Anat. 187: 271-277.

19. Lukomski. S., K. Nakashima, I. Abdi, V. J. Cipriano, R. M. Ireland, S. D. Reid, G. G. Adams, and J. M. Musser. 2001. Identification and characterization of the scl gene encoding a group A Streptococcus extracellular protein virulence factor with similarity to human collagen. Infect. Immun. 68: 6542-6553.

20. Lukomski. S., K. Nakashima, I. Abdi, V. J. Cipriano, B. J. Shelvin, E. A. Graviss, and J. M. Musser. 2001. Identification and characterization of a second extracellular collagen-like protein made by group A Streptococcus: control of production at the level of traslation. Infect. Immun. 69: 1729-1738.

21. Cunningham, M. W., S. M. Antone, J. M. Gulizia, B. A. McManus, V. A. Fischetti, and C. J. Gauntt. 1992. Cytotoxic and viral neutralizing antibodies cross react with streptococcal M protein, enteroviruses, and human cardiac myosin. Proc. Natl. Acad. Sci. USA. 89: 1320-1324

22. Quinn, A., T. M. Shinick and M. W. Cunningham. 1996. Anti-Hsp65 antibodies recognize M proteins of group A streptococci. Infect. Immun. 64: 818-824.

What is claimed is:

1. A cross-reactive monoclonal antibody which binds to a peptide selected from the group consisting of the A domain of the ACE protein from E. faecalis, ACE19 from E. faecalis and CNA19 from S. aureus.

2. The antibody according to claim 1 wherein said antibody treats or prevents infection from staphylococcal, streptococcal and enterococcal bacteria in a human or animal.

3. The antibody according to claim 1, wherein said antibody inhibits binding of staphylococcal, streptococcal or enterococcal bacteria to collagen.

4. The antibody according to claim 1, wherein said antibody is suitable for parenteral, oral, intranasal, subcutaneous, aerosolized or intravenous administration in a human or animal.

5. The antibody according to claim 1 wherein the monoclonal antibody is of a type selected from the group consisting of murine, chimeric, humanized and human monoclonal antibodies.

6. The antibody according to claim 1 wherein the antibody is a single chain monoclonal antibody.

7. The antibody according to claim 1 which comprises an antibody fragment having the same binding specificity of an antibody which binds to the E. faecalis ACE protein.

8. The antibody according to claim 1 that binds to a peptide having the sequence of from amino acids 32-567 in SEQ ID NO:1.

9. The antibody according to claim 8 wherein the peptide has an amino acid sequence encoded by a nucleic acid sequence according to nucleotides 94-1701 of SEQ ID NO:2, or degenerates thereof.

10. The antibody according to claim 1 that binds to a peptide having the sequence of from amino acids 174-319 in SEQ ID NO:1.

11. The antibody according to claim 1 that is selected from the group consisting of monoclonal antibodies 7E11, 8F1, 9D4, 10G1 and 11A6.

12. Isolated antisera containing an antibody according to claim 1.

13. A diagnostic kit comprising an antibody according to claim 1 and means for detecting binding by that antibody.

14. The diagnostic kit according to claim 13 wherein said means for detecting binding comprises a detectable label that is linked to said antibody.

15. A pharmaceutical composition for treating or preventing a bacterial infection comprising an effective amount of the antibody of claim 1 and a pharmaceutically acceptable vehicle, carrier or excipient.

16. The pharmaceutical composition according to claim 15 wherein the infection treated or prevented is selected from the group consisting of enterococcal, staphylococcal, and streptococcal bacteria.

17. A method of treating or preventing an infection of enterococcal, streptococcal, or staphylococcal infection comprising administering to a human or animal patient an effective amount of an antibody according to claim 1.

18. A method of inducing an immunological response comprising administering to a human or animal an immunogenic amount of an isolated protein from E. faecalis selected from the group consisting of the ACE40 protein and the ACE19 protein.

19. An isolated antibody according to claim 1 that has the ability to bind to the peptide of amino acids 32-567 of SEQ ID NO:1.

20. An isolated antibody according to claim 1 that has the ability to bind to an amino acid sequence coded by the nucleic acid sequence of nucleotides 94-1701 from SEQ ID NO:2 or degenerates thereof.

21. An isolated antibody according to claim 1 further comprising a physiologically acceptable antibiotic.

22. The antibody according to claim 1 wherein the antibody recognizes epitopes from bacteria selected from the group consisting of staphylococcal, enterococcal and streptococcal bacteria.


SEQUENCE LISTING (2 sequences)

SEQ ID NO: 1 - protein, 458 aa, Enterococcus faecalis
ELSKSSIVDKVELDHTTLYQGEMTSIKVSFSDKENQKIKPGDTITLTLPDELVGMTENDGSPRKINLNGLGEVFIYKDHVVATFNEKVESLHNVNGHFSFGIKTLITNSSQPNVIETDFGTATATQRLTIEGVTNTETGQIERDYPFFYKVGDLAGESNQVRWFLNVNLNKSDVTEDISIADRQGSGQQLNKESFTFDIVNDKETKYISLAEFEQQGYGKIDFVTDNDFNLRFYRDKARFTSFIVRYTSTITEAGQHQATFENSYDINYQLNNQDATNEKNTSQVKNVFVEGEASGNQNVEMPTEESLDIPLETIEEWEPKTPTSEQATETSEKTDTTETVESSQPEVHVSPTEEENPDESETLGTIEPILPEKPSVTTEENGTTETAESSQPEVHVSPTEEENPDESETLGIISPIIPEKPSVTTEENGTTETAESSQPEVHVSPTKEITTTEKKQP

SEQ ID NO: 2 - DNA, 1374 bp, Enterococcus faecalis
gaattgagcaaaagttcaatcgttgacaaagtagaattagatcacactactttatatcaaggggagatgacctccattaaagtatcttttagtgacaaagaaaatcagaaaataaaacctggcgatactattactttaactttaccagacgaactagttggaatgaccgagaacgatggctcaccacgaaaaatcaatttaaatggtttaggggaagtttttatctataaagatcatgttgtagcaacatttaatgaaaaagttgaatctttacataatgtgaatgggcatttttctttcgggattaaaacgcttatcaccaatagttcgcaaccgaatgtgatagaaacggatttcggaacagcaacggcgactcaacgtttgacgattgaaggagtgaccaacacagagactggccaaattgagcgagactatccgtttttttataaagtaggcgatttggctggagagtcaaatcaagtacgttggtttttaaatgtgaacctcaataaatccgatgtcacagaagatatttcaattgcggatcgacaaggaagtggtcaacaattaaataaagagagttttacatttgatattgtgaatgacaaagaaactaaatatatttcacttgccgagtttgagcaacaaggttatggcaaaattgacttcgtaacagataatgactttaatttacgtttttatcgggataaagcacgctttacttcctttatcgtccgttacacttcgacaatcacggaagcaggccaacatcaagcaacatttgaaaatagttatgacatcaattatcaactaaacaatcaagacgcaacgaatgaaaaaaatacatcacaggttaaaaatgtttttgtagaaggcgaggcaagcggcaatcaaaatgtggaaatgccaacagaagaaagtctagacattcctttagagacaatagaagaatgggaaccaaagacacctacttcggaacaggcaacagaaacaagtgaaaagacagacacaacagaaaccgtagaaagcagccaaccagaagttcatgtttcaccaacagaagaagaaaatccagatgaaagtgaaacactaggcacgattgagccaatcctacctgaaaaaccaagtgtgacaactgaagagaacggcacaacagaaaccgcagaaagcagtcaaccagaagttcatgtctcaccaacggaagaagaaaatccagatgaaagtgaaacgttaggtataatttcaccaattattcctgaaaaaccaagtgtgacaactgaagagaacggcacaacagaaaccgcagaaagcagtcaaccagaagtccatgtctcaccaacaaaagaaattactacaactgagaaaaaacagcca